(19) United States
(12) Patent Application Publication
(10) Pub. No.: US 2022/0219156 A1
(43) Pub. Date: Jul. 14, 2022

(54) CATALYST FOR THE CATALYTIC SYNTHESIS OF UREA

(71) Applicants: thyssenkrupp Industrial Solutions AG, Essen (DE); thyssenkrupp AG, Essen (DE)

(72) Inventors: Christoph GLOTZBACH, Speyer (DE); Nils TENHUMBERG, Dortmund (DE); Tarek EL HAWARY, Holzwickede (DE); Yevgeny MAKHYNYA, Mülheim an der Ruhr (DE); Walter LEITNER, Aachen (DE); Jürgen KLANKERMAYER, Essen (DE); Hannah SCHUMACHER, Köln (DE)

(73) Assignees: thyssenkrupp Industrial Solutions AG, Essen (DE); thyssenkrupp AG, Essen (DE)

(21) Appl. No.: 17/607,756

(22) PCT Filed: Apr. 27, 2020
(86) PCT No.: PCT/EP2020/061615

§ 371 Date: Oct. 29, 2021

(30) Foreign Application Priority Data
Apr. 29, 2019 (DE) 10 2019 111 060.2

(51) Int. Cl. B01J 31/24; C07C 273/02

(52) U.S. Cl. CPC B01J 31/24; C07C 2531/24; C07C 273/02

(57) ABSTRACT

A ruthenium-phosphine complex can be used as a catalyst in a method for the catalytic synthesis of urea. The method may comprise more particularly a reaction of formamide or of formamide with ammonia in the presence of the catalyst to form urea and hydrogen. Through the use of the ruthenium-phosphine complex as the catalyst, catalytic preparation of urea from formamide or from formamide with ammonia is provided for the first time. This allows for synthesis under mild conditions and virtually no formation of byproducts. Further, using an acid as a cocatalyst in the catalytic synthesis or the reaction can lead to an improvement in urea yield.

The invention relates to a ruthenium catalyst for the catalytic synthesis of urea.

Urea, the diamide of carbonic acid, is one of the most important bulk chemicals and is used predominantly as fertilizer. As such it possesses a high nitrogen content (46 wt %). It is easily hydrolyzed, releasing ammonia and CO₂, by the enzyme urease, which is produced by microorganisms and occurs widely in the soil.

Furthermore, urea is an important building block for organic products, such as melamine, and a raw material for synthetic resins and fibers. It is used as a cattle feed additive and in the production of drugs and explosives, and in the textile industry as well. In recent decades, urea has also gained importance as a reducing agent for the NOx reduction of diesel exhaust gases.

Urea is produced industrially almost exclusively in a high-pressure synthesis from ammonia (NH₃) and carbon dioxide (CO₂) at about 150 bar and about 180° C. The two reactants generally come from an ammonia plant, which is usually situated in the close vicinity of a urea plant.

In this high-pressure synthesis, CO₂ separated off in advance is brought into association with liquid ammonia. In the first step of the synthesis, ammonium carbamate is synthesized primarily. During the course of the reaction, urea as well is formed in small quantities, to produce a complex mixture composed of ammonia, CO₂, urea, ammonium carbamate, ammonium hydrogencarbonate, and water. This takes place in an apparatus referred to as a carbamate condenser. The reaction mixture departs the carbamate condenser for the urea reactor, where the actual urea formation reaction occurs. Because the carbamate is a highly corrosive medium, a particularly corrosion-resistant steel is required at many points in the process, which is extremely costly and massively increases the capital costs of the plant. Not only the steel but also the high-pressure and high-temperature operation impose a major challenge for the apparatuses in the high-pressure circuit, this being ultimately reflected in the acquisition costs of these apparatuses.

Alternative routes to urea are the reaction of ammonia with phosgene (see D. Roeda et al., Int. J. Appl. Radiat. Isot. 1980, 31, 549-551), cyanide (see A. M. Emran et al., Int. J. Appl. Radiat. Isot. 1983, 34, 1013-1014) or with carbon monoxide in the presence of sulfur or selenium as oxidizing agent (see, for example, K. Kondo et al., Angew. Chem. 1979, 91, 761-761). These routes, however, require the use of highly toxic reactants and produce stoichiometric amounts of byproducts. A catalytic route to urea is therefore highly desirable.

Substituted urea derivatives can be prepared catalytically via various routes, using CO and CO₂ or other carbonylating agents. The synthesis of substituted urea derivatives by means of CO is described for example in D. J. Diaz et al., Eur. J. Org. Chem. 2007, 2007, 4453-4465. The synthesis of substituted urea derivatives by means of CO₂ is described for example in P. Munshi, et al., Tetrahedron Lett. 2003, 44, 2725-2727. The synthesis with other carbonylating agents is reported for example in A. Basha, Tetrahedron Lett. 1988, 29, 2525-2526.

Relative to the incorporation of amines for substituted ureas, additional challenges exist however when using ammonia to prepare urea, since ammonia has three potentially active hydrogens and a significantly different basicity. Consequently there are only relatively few publications which report on catalytic synthesis of urea, examples including M. M. Taqui Khan, S. B. Halliqudi, S. H. R. Abdi, S. Shukla, J. Mol. Catal. 1988, 48, 25-27; D. C. Butler, D. J Cole-Hamilton, Inorg. Chem. Commun. 1999, 2, 305-307; F. Barzagli et al., Green Chem. 2011, 13, 1267-1274; A. R. Elman, V. I. Smirnov, J. Environ. Sci. Eng. 2011, 5, 1006-1012.

Ammonia is the usual starting material in the synthesis of urea. Furthermore, CO₂ is a readily available feedstock for urea synthesis. In the search for a catalytic route to the synthesis of urea based on CO₂, the starting point contemplated was a two-stage process via formamide as intermediate, as depicted in scheme 1:

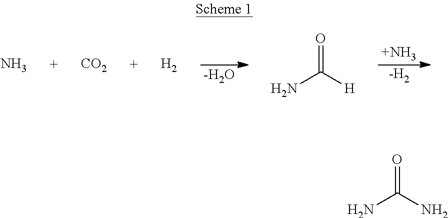

While syntheses of substituted urea from formamides have been described for example in S. Kotachi, Y. Tsuji, T. Kondo, Y. Watanabe, J. Chem. Soc., Chem. Commun. 1990, 549-550, the formation of urea from the reaction of formamide with ammonia represents a new and challenging C—N bond formation.

The object on which the invention is based is that of providing a catalyst for the catalytic synthesis of urea in order to overcome the above-described disadvantages of the conventional noncatalytic processes, more particularly for a synthesis based on formamide as starting material. The object more particularly, through the provision of a suitable catalyst for the urea synthesis, is that of reducing or entirely avoiding the formation of byproducts, such as of ammonium carbamate, for example. The reaction is to be able to be carried out under extremely mild pressure and temperature conditions and the catalyst is to have a high catalytic productivity. The plants required for the synthesis with the catalyst are to be extremely simple and inexpensive.

Surprisingly the inventors have achieved this through the use of a specific catalyst system. Provided accordingly is a system for the synthesis of urea using a specific ruthenium catalyst. Starting materials used for the synthesis were, in particular, formamide, or formamide and ammonia.

This object is therefore achieved in accordance with the invention by means of the use as claimed in claim 1. Further preferred embodiments of the use according to the invention are set out in the dependent claims.

As a result of the catalyst used in the invention it is possible to prepare urea, more particularly from formamide or from formamide and ammonia, catalytically under mild conditions, with hydrogen being formed as a coproduct. Where formamide is reacted in the absence of added ammonia, CO is additionally formed. Virtually no byproducts are formed. The hydrogen liberated in the reaction can be reused for the synthesis of formamide.

The invention and preferred embodiments thereof are elucidated in detail below.

The invention relates to the use of a ruthenium-phosphine complex as catalyst for the catalytic synthesis of urea, where the synthesis preferably comprises the reaction of formamide or of formamide with ammonia in the presence of the ruthenium-phosphine complex as catalyst to form urea and hydrogen.

In the case of the use of the ruthenium-phosphine complex in the invention as catalyst for the catalytic synthesis of urea, the synthesis preferably comprises the reaction of formamide with ammonia in the presence of the ruthenium-phosphine complex as catalyst to form urea and hydrogen. In the case of the use of the ruthenium-phosphine complex in the invention as catalyst for the catalytic synthesis of urea, an alternative synthesis comprises the reaction of formamide in the presence of the ruthenium-phosphine complex as catalyst to form urea and hydrogen, with CO as well being formed in the case of this alternative. In the alternative variant, only formamide is used as starting material for the catalytic synthesis or reaction in the presence of the ruthenium-phosphine complex as catalyst to form urea; in particular, no NH₃ is added to the reaction mixture. Starting materials used for the synthesis are therefore formamide or, preferably, formamide and ammonia.

Unless indicated otherwise, the elucidations relating to the use of the ruthenium-phosphine complex as catalyst for the catalytic synthesis of urea refer both to the preferred variant and to the alternative variant, as have been indicated above. It will be appreciated that details relating to the added ammonia refer only to the preferred variant.

The preparation of urea by reaction of formamide with ammonia, using the catalyst of the invention, may be illustrated by the following equation:

The ruthenium-phosphine complex comprises one or more phosphine ligands. The

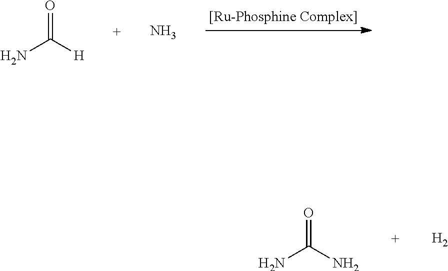

phosphine may be a simple phosphine (monophosphine), a compound having two phosphine groups (diphosphine), a compound having three phosphine groups (triphosphine), or a compound having more than three phosphine groups.

The phosphines are, in particular, trivalent organophosphorus compounds. The phosphine is more particularly a tertiary phosphine or has two, three or more tertiary phosphine groups. The phosphine is, for example, a compound PR¹R²R³, in which R¹, R² and R³ independently of one another each represent an organic radical. The substituents R¹, R² and R³ are preferably independently of one another each substituted or unsubstituted alkyl, substituted or unsubstituted aryl or substituted or unsubstituted heteroaryl.

Identified below are suitable and preferred examples of the groups alkyl, aryl and heteroaryl, and also suitable examples of substituents of corresponding substituted groups, which are valid as examples for all of the references in the present application to these groups or substituted groups, unless explicitly excluded. The examples of the groups alkyl, aryl and heteroaryl are also examples of these groups when they are present as substituents of a group.

Alkyl here also includes cycloalkyl. Examples of alkyl are linear and branched C₁-C₈ alkyl, preferably linear and branched C₁-C₆ alkyl, e.g. methyl, ethyl, n-propyl, isopropyl or butyl and C₃-C₈ cycloalkyl.

Substituted alkyl may have one or more substituents, e.g. halide, such as chloride or fluoride, aryl, heteroaryl, cycloalkyl, alkoxy, e.g. C₁-C₆ alkoxy, preferably C₁-C₄ alkoxy, or aryloxy. Unsubstituted alkyl is preferred.

Examples of aryl are selected from homoaromatic compounds having a molecular weight below 300 g/mol, preferably phenyl, biphenyl, naphthalenyl, anthracenyl and phenanthrenyl.

Examples of heteroaryl are pyridinyl, pyrimidinyl, pyrazinyl, triazolyl, pyridazinyl, 1,3,5-triazinyl, quinolinyl, isoquinolinyl, quinoxalinyl, imidazolyl, pyrazolyl, benzimidazolyl, thiazolyl, oxazolidinyl, pyrrolyl, carbazolyl, indolyl and isoindolyl, where the heteroaryl may be joined to the phosphorus group of the phosphine via any desired atom in the ring of the selected heteroaryl. Preferred examples are pyridinyl, pyrimidinyl, quinolinyl, pyrazolyl, triazolyl, isoquinolinyl, imidazolyl and oxazolidinyl, where the heteroaryl may be joined to the phosphorus group of the phosphine via any desired atom in the ring of the selected heteroaryl.

Substituted aryl and substituted heteroaryl may have one, two or more substituents. Examples of suitable substituents for aryl and heteroaryl are alkyl, preferably C₁-C₄-alkyl, e.g. methyl, ethyl, n-propyl or isopropyl, perfluoroalkyl, e.g. —CF₃, aryl, heteroaryl, cycloalkyl, alkoxy, e.g. C₁-C₆ alkoxy, preferably C₁-C₄ alkoxy, aryloxy, alkenyl, e.g. C₂-C₆ alkenyl, preferably C₃-C₆ alkenyl, silyl, amine and fluorene. Preference is given to unsubstituted aryl, more particularly phenyl, and unsubstituted heteroaryl.

According to one preferred embodiment the phosphine in the ruthenium-phosphine complex is PR¹R²R³, in which R¹, R² and R³ independently of one another are substituted or unsubstituted heteroaryl or substituted or unsubstituted aryl, more particularly phenyl, e.g. tri(heteroaryl)phosphine or tri(aryl)phosphine, or a PR¹R²R³, in which R¹ is alkyl and R² and R³ independently of one another are substituted or unsubstituted heteroaryl and/or substituted or unsubstituted aryl, more particularly phenyl, e.g. di(heteroaryl)alkylphosphine or di(aryl)alkylphosphine.

More preferably the phosphine in the ruthenium-phosphine complex is a compound having two phosphine groups (diphosphine), a compound having three phosphine groups (triphosphine) or a compound having more than three phosphine groups, the phosphine more preferably being a triphosphine. The phosphines having two or more phosphine groups derive preferably from two or more identical or different phosphines PR¹R²R³ as described above, with at least one substituent of the phosphines being linked to one or more other substituents of the phosphines to form a joint group, such as an alkylene group with a valence of two, three or more, as a bridging unit. The details above concerning the substituents and preferred substituents/phosphines are valid analogously for the compounds having more than one phosphine group.

According to one preferred embodiment of the present invention the ruthenium-phosphine complex contains more than one phosphine group, meaning that there are two or more monophosphines, at least one diphosphine or triphosphine, or a compound having more than three phosphine groups, as ligands in the coordination sphere of the ruthenium.

The bonds between the ruthenium and the phosphine group are formed at least temporarily during the reaction, e.g. a covalent or coordinative bond. It should be noted that in the case of the reaction according to the invention in the presence of the ruthenium-phosphine complex, not all phosphines/phosphine groups in the reaction mixture are necessarily bonded to the ruthenium. In fact the phosphine may be used in excess, meaning that unbonded phosphines/phosphine groups may also be present in the reaction mixture. Particularly if compounds having more than three phosphine groups are used, it is generally the case that not all of the phosphorus atoms are involved catalytically in the reaction; nevertheless, these compounds are also preferred compounds within the present invention.

Particularly preferred are ruthenium-triphosphine complexes where the bridging unit between the phosphorus atoms in the triphosphine is an alkyl or alkylene unit, while the further ligands are heteroaryl with or without substitution or aryl with or without substitution on the phosphorus.

According to one preferred embodiment of the present invention, the ruthenium-triphosphine complex comprises a triphosphine of the general formula I

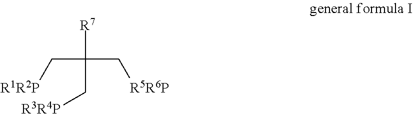

where R¹ to R⁶ independently of one another are substituted or unsubstituted aryl or substituted or unsubstituted heteroaryl, preferably substituted or unsubstituted aryl, and R⁷ is hydrogen or an organic component, preferably alkyl, cycloalkyl or aryl. Examples of suitable substituents for aryl and heteroaryl have been stated above, preference being given to alkyl, more particularly methyl, ethyl, n-propyl, isopropyl, alkoxy, for example methoxy, and perfluoroalkyl, for example —CF₃. The substituted or unsubstituted aryl is preferably unsubstituted aryl, more particularly phenyl. The substituted or unsubstituted heteroaryl is preferably unsubstituted heteroaryl.

The substituents R¹ to R⁶ may be identical or different, and are preferably identical. More preferably R¹ to R⁶ are substituted or unsubstituted phenyl. The substituted aryl, more particularly substituted phenyl, may have one, two or more substituents, in ortho- and/or para-position, for example. Examples of suitable substituents have been stated above, preference being given to alkyl, more particularly methyl, ethyl, n-propyl, isopropyl, alkoxy, such as methoxy, or perfluoroalkyl, such as —CF₃. With particular preference R⁷ is an alkyl, more preferably methyl or ethyl, more particularly methyl.

One particularly preferred phosphine ligand for the ruthenium-phosphine complex is 1,1,1-tris(diphenylphosphinomethyl)ethane (triphos), which has the following structure:

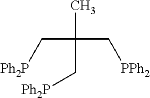

Besides the aforementioned phosphine ligand or ligands, the ruthenium-phosphine complex may have one or more further ligands (nonphosphine ligands), such as, for example, carbenes, amines, amides, phosphites, phosphoamidites, phosphorus-containing ethers or esters, sulfides, trimethylenemethane, cyclopentadienyl, allyl, methylallyl, ethylene, cyclooctadiene, acetylacetonate, acetate, hydride, halide, such as chloride, phenoxide or CO, particularly if the ruthenium-phosphine complex comprises an above-described diphosphine, triphosphine or a compound having more than three phosphine groups.

The one or more further ligands are preferably selected from trimethylenemethane, cyclopentadienyl, allyl, methylallyl, ethylene, cyclooctadiene, acetylacetonate, acetate, hydride, halide, phenoxide, CO or a combination thereof, particular preference being given to trimethylenemethane (tmm). These ligands have a labile bond to ruthenium, and so can easily be substituted by reactant species during the catalytic reaction sequence. Furthermore, a catalyst precursor can be stabilized with these ligands.

In one preferred embodiment the ruthenium-phosphine complex has the following general formula II:

(A)Ru(L)₃ general formula II

in which A is a triphosphine of the general formula I as defined above and L independently of one another in each case are monodentate ligands, it being possible for two monodentate ligands L to be replaced by one bidentate ligand or for three monodentate ligands L to be replaced by one tridentate ligand. Examples of the mono-, bi- or tridentate ligands L are the above-stated further ligands (nonphosphine ligands), in which case they are preferably selected from trimethylenemethane, cyclopentadienyl, allyl, methylallyl, ethylene, cyclooctadiene, acetylacetonate, acetate, hydride, halide, phenoxide, CO or a combination thereof, particular preference being given to trimethylenemethane (tmm). The ligand tmm is a tridentate ligand, for example.

One particularly preferred ruthenium-triphosphine complex has the following structure:

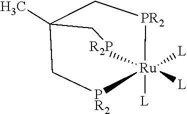

where the substituents R in each case independently of one another are substituted or unsubstituted aryl or substituted or unsubstituted heteroaryl, preferably substituted or unsubstituted aryl, and L in each case independently of one another are monodentate ligands, it being possible for two monodentate ligands L to be replaced by one bidentate ligand or for three monodentate ligands L to be replaced by one tridentate ligand. Examples of suitable substituents for aryl and heteroaryl have been stated above, preference being given to alkyl, more particularly methyl, ethyl, n-propyl, isopropyl, alkoxy, e.g. methoxy, and perfluoroalkyl, such as —CF₃. The substituted or unsubstituted aryl is preferably unsubstituted aryl, more particularly phenyl. The substituted or unsubstituted heteroaryl is preferably an unsubstituted heteroaryl.

The substituents R may be identical or different, and are preferably identical. More preferably R is substituted or unsubstituted phenyl. The substituted phenyl may have one, two or more substituents, especially in ortho- and/or para-position. Examples of suitable substituents have been given above, preference being given to alkyl, more particularly methyl, ethyl, n-propyl, isopropyl, alkoxy, e.g. methoxy, and perfluoroalkyl, such as —CF₃. The triphosphine ligand is more preferably triphos.

Examples of the mono-, bi- or tridentate ligands L are the above-stated further ligands (nonphosphine ligands), these ligands being preferably selected from trimethylenemethane, cyclopentadienyl, allyl, methylallyl, ethylene, cyclooctadiene, acetylacetonate, acetate, hydride, halide, phenoxide, CO or a combination thereof, particular preference being given to trimethylenemethane (tmm).

One particularly preferred ruthenium-phosphine complex is [Ru(triphos)(tmm)] with the following structural formula:

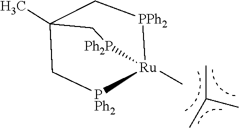

The ruthenium-phosphine complexes identified above are known and may be prepared by the skilled person in accordance with known methods, and/or are available commercially. [Ru(triphos)(tmm)] is described for example in T. vom Stein et al., ChemCatChem 2013, 5, 439-441.

The ruthenium-phosphine complex may also be prepared in situ in the reaction mixture for the reaction. The preparation of the ruthenium-phosphine complex in situ is possible from catalyst precursors, the phosphines, more particularly triphosphines, and optionally further ligands. Examples of catalyst precursors employed for this purpose are Ru(acac)₃, Ru(cod)(methylallyl)₂, Ru(nbd)(methylallyl)₂ and Ru(ethylene)₂(methylallyl)₂ where acac=acetylacetonate, cod=1,5-cyclooctadiene and nbd=norbornadiene.

The ruthenium-phosphine complex may be used as a homogeneous catalyst or as an immobilized catalyst in the catalytic reaction of formamide or of formamide and ammonia to give urea. Two-phase systems with phase transfer catalysis are also possible. The catalytic reaction with the ruthenium-phosphine complex, may be carried out homogeneously or heterogeneously, with, for example, an immobilized catalyst in a fixed bed reactor or a dissolved catalyst in a fluidized bed reactor.

The catalytic synthesis of urea, more particularly the catalytic reaction of formamide or of formamide and ammonia may be carried out continuously or batchwise, with continuous operation being preferred. The catalytic synthesis or catalytic reaction is carried out preferably in an autoclave or a pressure reactor. An autoclave is suitable for batch operation. A pressure reactor is suitable for continuous operation.

The catalytic synthesis of urea, more particularly the catalytic reaction of formamide or of formamide and ammonia may optionally be carried out, additionally, in the presence of an acid as cocatalyst, and the acid in question may be a Brønsted acid or a Lewis acid. The acid may be an organic acid or an inorganic acid. This acid may lead to the additional activation of the catalyst and/or the formamide, and may improve the yield of the reaction.

Examples of judicious Brønsted acids or Lewis acids are organoaluminum compounds, such as aluminum triflate (aluminum tris(trifluoromethanesulfonate)) and aluminum triacetate, organoboron compounds, such as tris(pentafluorophenyl)borane, sulfonic acids, such as p-toluenesulfonic acid, bis(trifluoromethane)sulfonimide (HNTf₂), scandium compounds, such as scandium triflate, perfluorinated copolymers containing at least one sulfo group, of the kind obtainable under the trade name Nafion® NR50, for example, or combinations thereof.

The catalytic synthesis of urea, more particularly the catalytic reaction of formamide or the catalytic reaction of formamide and ammonia to give urea takes place for example at a temperature in the range from 50 to 250° C., preferably in the range from 120 to 200° C., more preferably in the range from 140 to 170° C.

The catalytic synthesis of urea, more particularly the catalytic reaction of formamide or of formamide and ammonia to give urea takes place for example at a pressure (reaction pressure) in the range from ambient pressure to 150 bar, preferably in the range from 2 bar to 60 bar, more preferably in the range from 5 to 40 bar. In the case of the preferred variant, the reaction may take place optionally under conditions in which liquid or supercritical ammonia is present (critical pressure (NH₃)=113 bar; critical temperature (NH₃)=132.5° C.), which can act as solvent.

In the preferred variant, the amount of ammonia used in the reaction, in equivalents (eq) based on formamide, may be for example in the range from 1 to 300 eq, preferably from 4 eq to 100 eq, more preferably from 29 to 59 eq.

In one preferred embodiment the reaction takes place with about 29 to 59 eq of ammonia, based on formamide, at a pressure in the range from 5 to 40 bar, preferably 10 to 30 bar. Solvents employed with particular preference in this case are dioxane, more particularly 1,4-dioxane, or toluene.

The reaction preferably takes place, accordingly, with a high stoichiometric excess of ammonia. This enables an improvement in the yield of urea.

The suitable reaction time for the catalytic synthesis of urea, more particularly the catalytic reaction of formamide or preferably of formamide with ammonia may vary depending on the other reaction parameters. The reaction time of the reaction is situated judiciously, for example, in a range from 1 minute to 24 hours or 30 minutes to 24 hours, preferably 3 to 15 hours, more preferably 6 to 10 hours.

In the use of the invention, the catalytic synthesis of urea, more particularly the catalytic reaction of formamide or of formamide with ammonia may be carried out in the absence or presence of solvent, more particularly organic solvent. In the absence of solvent, an optional excess of ammonia in the form of liquid or preferably supercritical ammonia may act as solvent.

In one preferred embodiment the catalytic synthesis of urea, more particularly the catalytic reaction, is carried out in a solvent, more particularly an organic solvent. One solvent or a mixture of two or more solvents may be employed, with preference being given to the use of one solvent.

The solvent is preferably an organic solvent, more particularly an aprotic organic solvent. The solvent may be polar or nonpolar, with nonpolar organic solvents being preferred. The solvent is preferably selected such that the ruthenium-phosphine complex used can be at least partly dissolved therein.

The solvent is preferably selected from the group consisting of cyclic and noncyclic ethers, substituted and unsubstituted aromatics, alkanes and halogenated hydrocarbons, such as trichloromethane, for example, and alcohols, with the solvent being selected preferably from halogenated hydrocarbons, cyclic ethers and substituted or unsubstituted aromatics, preferably from cyclic ethers and substituted or unsubstituted aromatics. Examples of aromatics are benzene or benzene having one or more aromatic substituents (e.g. phenyl) and/or aliphatic substituents (e.g. C₁-C₄ alkyl). Particularly preferred solvents are dioxane, more particularly 1,4-dioxane, toluene, and tetrahydrofuran (THF). However, dichloromethane or trichloromethane may also be used with advantage.

As solvents it is optionally possible alternatively to use ionic liquids as well. Ionic liquids are known to the skilled person. These are salts which are liquid at low temperatures, such as at temperatures of not more than 100° C. The cation of the ionic liquid is selected, for example, from imidazolium, pyridinium, pyrrolidinium, guanidinium, uronium, thiouronium, piperidinium, morpholinium, ammonium and phosphonium, and this cation may be substituted preferably by one or more alkyl groups. The anion of the ionic liquid is selected, for example, from halides, tetrafluoroborates, trifluoroacetates, triflates, hexafluorophosphates, phosphinates, tosylates or organic ions, such as imides or amides, for example.

The ruthenium-phosphine complex is present preferably at least partly or completely in solution in the solvent. The catalytic synthesis of urea, more particularly the catalytic reaction of formamide or of formamide with ammonia to give urea is preferably a homogeneous catalytic reaction. Catalyst and reactants here are present in solution, in other words in the same phase. The homogeneous catalysis may enable milder reaction conditions and possibly higher selectivities and higher turnover numbers (TON) and/or turnover frequency (TOF).

The concentration of the one or more solvents is situated, for example, in a range from 5 to 500 mL, preferably from 10 to 300 mL, more preferably from 50 to 250 mL, per 1 mmol of Ru-phosphine complex.

The concentration of ruthenium-phosphine complex as catalyst in the reaction may be situated, for example, in the range from 0.05 mol % to 10 mol %, preferably from 0.25 mol % to 5 mol %, more preferably 0.5 mol % to 2 mol %, based on the molar amount of formamide.

Since during preparation the ruthenium-phosphine complexes are generally sensitive to air and to moisture, they are preferably prepared very largely in the absence of air and moisture, for which the conventional methods such as Schlenk technologies and operations in a glovebox are employed. Reaction apparatus, such as glass equipment, for example, and reagents employed are where necessary dried and/or freed from air in accordance with conventional techniques.

The catalytic reaction of formamide or of ammonia and formamide takes place usefully, though not necessarily, in an inert gas atmosphere or very largely to the exclusion of oxygen, since this minimizes any oxidation of the catalyst. Nitrogen is an example of a suitable inert gas for this purpose. The exclusion of oxygen is especially useful when the hydrogen liberated in the reaction is to be returned to the NH₃ plant and used therein for the synthesis of urea and/or NH₃. The catalyst used in the NH₃ synthesis is sensitive to oxygen, and so the insertion of additional oxygen must be avoided.

There are different possible uses for the hydrogen formed in the reaction according to the invention: it may be used, in fact, for energy or as the element in a downstream plant, such as in an ammonia synthesis plant, for instance an ammonia plant of the ammonia-urea complex, in which these compounds are produced in an integrated system.

Generally speaking, the reaction mixture obtained from the above-described catalytic reaction of formamide or of formamide and ammonia is processed in order to recover the urea formed and to recycle the remaining reactants, catalyst and optionally solvent. For this purpose it is possible to implement processing steps which are customary in the prior art and in the industry, such as gas-liquid separation, filtration, etc. The product streams obtained in the processing therefore include a gas stream, consisting predominantly of hydrogen and ammonia, and a liquid stream, which comprises urea, catalyst, residues of formamide, and any solvent. The gas stream may be recovered from the resultant reaction mixture at elevated temperature, this being advantageous for subsequent re-use, as there is no need for the gases to be compressed again. Compressed gas is generally needed in possible uses of the gases, such as for the synthesis of urea and/or NH₃, for example.

For the processing, the pressurized reaction mixture is subjected preferably to a gas-liquid separation, without draining pressure from the reaction mixture. This separation may take place with or without prior cooling of the reaction mixture.

The processing generally comprises the removal of hydrogen formed and of unreacted ammonia in gas form, this taking place generally in the ammonia plant; the cooling of the remaining liquid residue to a temperature of below 0° C.; and then the filtration or centrifugation of the residue, giving urea as a solid. The urea obtained in solid form is then freed from residues of catalyst and formamide, generally by washing with a solvent, and is then subjected to granulation. Granulation in the present patent application refers to any form of compacting, unless otherwise indicated.

An advantage of the use according to the invention is that no biuret is formed from urea, meaning that processing residues containing traces of urea can be recycled as desired.

The gases can be isolated from the reaction mixture conventionally. For more effective isolation of the gases (hydrogen/ammonia), it is possible optionally to use a gas such as nitrogen as a stripping agent. As a result of the reaction mixture being stripped with nitrogen, the gaseous components can be expelled more effectively. Processing of the gas stream obtained allows ammonia to be isolated, and it can be returned to the urea synthesis or used for the formamide synthesis. The nitrogen/hydrogen mixture that is left may be returned as syngas makeup to the ammonia synthesis or formamide synthesis.

The liquid reaction residue obtained after removal of gases typically contains urea, catalyst, excess formamide and traces of ammonia, and also, possibly, solvent. The urea contained in the reaction residue is partly precipitated even at room temperature. In order to maximize precipitation, it is advantageous to cool the reaction residue to low temperatures. The reaction residue is cooled down preferably to a temperature of below 0° C., more preferably below at least −10° C. or at least −20° C., e.g. down to about −30° C. At these low temperatures, urea is very largely precipitated. Even greater cooling to temperatures below −30° C. is also possible, although in that case it is necessary to weigh economic factors, such as cooling costs, against improved yield.

Thereafter the solid is removed from the reaction residue, by filtration or centrifugation, for example. The solid removed contains primarily urea and traces of solvent, formamide and catalyst. The solid obtained may then be cleaned by washing with solvent and subjected to granulation, to give the urea as a finished product.

The liquid residue which remains when the solid has been separated from the reaction residue, this liquid residue generally being the filtrate or centrifugate, is combined in general with the wash solution used for washing the solid. The resulting mixture typically contains solvent, catalyst, residues of formamide and traces of urea. The mixture obtained may simply be passed back to the reaction and combined with the makeup or starting material for the reaction of formamide, preferably ammonia. As indicated above, no biuret is formed from urea, and so the mixture containing traces of urea can be recycled as desired.

Alternatively, excess solvent from the downstream washing of the solid with solvent may be removed from the resultant mixture by distillation and recycled if of sufficient quality. Following removal, the formamide may be passed back into the reaction. The catalyst may optionally be reused in the process. If the catalyst is deactivated, the remaining residue may optionally be subjected to recrystallization beforehand, in order to separate urea and catalyst from one another and to subject the catalyst to a regeneration.

### EXAMPLES

### Synthesis of [Ru(Triphos)(Tmm)]

A 35 mL Schlenk tube was filled with 319 mg (1.00 mmol) of [Ru(cod)(methylallyl)] (cod=1,5-cyclooctadiene) and 624 mg (1.17 mmol) of 1,1,1-tris(diphenyl-phosphinomethyl)ethane in 20 mL of toluene. The reaction mixture was stirred and was heated at 110° C. for 2 h, cooled to room temperature and concentrated under reduced pressure. Following treatment with 15 mL of pentane, the precipitating complex was isolated, washed with pentane (3×10 mL) and dried under reduced pressure overnight, to give [Ru(triphos)(tmm)] as a pale yellow powder (0.531 g, 0.678 mmol, 68% yield). The identity was confirmed by ¹H, ¹³C APT and ³¹P NMR spectra.

### Examples 1-9

Synthesis of Urea from Formamide and Ammonia with Ru(Triphos)(Tmm)

The urea was synthesized in accordance with the following equation:

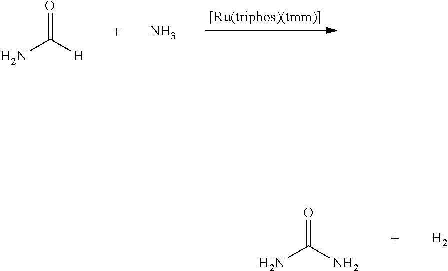

High-pressure batch experiments were performed in a 10 mL stainless steel autoclave fitted with a glass insert and a magnetic stirring rod. When 2 mL of 1,4-dioxane and 0.6 g of NH₃ were used, the reaction pressure was about 30 bar in the hot state (at 150° C. reaction temperature) and the pressure in the cold state (room temperature) was about 8-10 bar. Before being used, the autoclave was evacuated for at least 30 minutes and filled repeatedly with argon. The catalyst [Ru(triphos)(tmm)] (7.8 mg, 0.01 mmol) was weighed under an argon atmosphere into a Schlenk tube and dissolved in 1,4-dioxane (2.0 mL). Following addition of formamide (40 μL, 1.00 mmol), the reaction mixture was transferred to the autoclave with a cannula under an argon countercurrent. Liquid NH₃ (between 0.5 g and 1.0 g) was introduced into the autoclave, and the autoclave was sealed. The reaction mixture was stirred and was heated to the respective reaction temperature in an aluminum cone for the respective reaction time. After cooling to room temperature, the autoclave was cautiously let down with air. Following removal of the solvent under reduced pressure, the reaction solution obtained was analyzed by ¹H and ¹³C NMR spectroscopy, using mesitylene as internal standard, and the yield of urea relative to formamide was determined.

The experiment was repeated a number of times, with the catalyst loading, solvent, reaction temperature and reaction time being varied as shown in table 1 below. Table 1 also shows the yield of urea obtained.

The catalyst loading is the amount of catalyst used in mol %, relative to the amount of formamide used (in mol).

TABLE 1
Ru-catalyzed synthesis of urea from formamide and ammonia*

| | Catalyst | | Reaction | Reaction | |
|---|---|---|---|---|---|
| | loading | | temperature | time | |
| Ex. | [mol %] | Solvent | [° C.] | [hours] | Yield |
| 1 | 1.00 | 1,4-Dioxane | 150 | 5 | 44 |
| 2 | 1.00 | 1,4-Dioxane | 150 | 10 | 64 |
| 3 | 1.00 | 1,4-Dioxane | 150 | 15 | 57 |
| 4 | 1.00 | 1,4-Dioxane | 130 | 10 | 12 |
| 5 | 1.00 | 1,4-Dioxane | 110 | 10 | 1 |
| 6 | 0.50 | 1,4-Dioxane | 150 | 10 | 26 |
| 7 | 0.25 | 1,4-Dioxane | 150 | 10 | 14 |
| 8 | 1.00 | Toluene | 150 | 10 | 53 |
| 9 | 1.00 | THF | 150 | 10 | 47 |

*Reaction conditions: [Ru(triphos)(tmm)], 1 mmol formamide, 2 mL solvent, 0.5-1.0 g NH₃

### Example 10

### Preparation of Ru(Triphos)(Tmm) In Situ for Synthesis of Urea

The catalyst Ru(triphos)(tmm) was formed in situ from the catalyst precursor [Ru(cod)(methylallyl)₂] and triphos.

For this, 1 mol % of [Ru(cod)(methylallyl)₂], 1.3 mol % of triphos, 1 mmol of formamide, 2 mL of 1,4-dioxane and 0.6 g of NH₃ were reacted at 150° C. for 10 h. The pressure was about 8 bar in the cold state and about 30 bar at 150° C. The yield of urea was 51%.

### Example 11

Synthesis of Urea from Formamide in the Absence of Ammonia

1 mol % of [Ru(triphos)tmm], 1 mmol of formamide and 2 mL of 1,4-dioxane were reacted at 150° C. and 15 bar for 10 h. The yield of urea was 7%.

### Examples 12 to 18

### Catalytic Activity of Ru-Phosphine Complexes as a Function of the Ligands on the Phosphorus

The catalytic activity of various Ru-phosphine complexes in the synthesis of urea from formamide and ammonia was tested as a function of the ligands on the phosphorus. Table 2 indicates the complexes (catalysts) studied, the reaction conditions and the yields obtained. In the experiments the reaction pressure was about 30 bar at the reaction temperature and the pressure in the cold state was about 8 bar, except in ex. 15.

Ruthenium-triphosphine complexes with the following structure were studied:

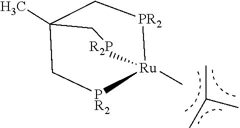

The nature of the substituent R is shown in table 2 below; where not all of the substituents R on the three phosphorus atoms are the same, the substituents R on a first P atom are identified as R¹, on a second P atom as R², and on a third P atom as R³. For example, the complex of ex. 17 has two phenyl groups on two phosphine groups, and the third phosphine group has two isopropyl groups.

The ruthenium-triphosphine complex additionally possesses the tridentate ligand trimethylenemethane.

The pressures reported in the table relate to room temperature (about 23° C.). The autoclave was charged at room temperature and then brought to reaction temperature and reaction pressure.

TABLE 2

| | | | Urea |
|---|---|---|---|
| Ex. | R = | Reaction conditions | yield |
| 12 | 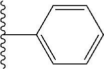 [SMILES: *c1ccccc1] | 1 mol % cat., 1 mmol formamide, 2 mL 1,4- dioxane, 0.6 g NH₃, 150° C., 10 h | 64% |
| 13 | 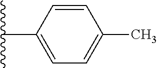 [SMILES: *c1ccc(C)cc1] | 0.5 mol % cat., 1 mmol formamide, 2 mL 1,4- dioxane, 8 bar NH₃, 150° C., 20 h | 8% |
| 14 | 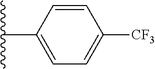 [SMILES: *c1ccc(C(F)(F)F)cc1] | 0.5 mol % cat., 1 mmol formamide, 2 mL 1,4- dioxane, 8 bar NH₃, 150° C., 20 h | 8% |
| 15 | 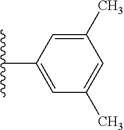 [SMILES: *c1cc(C)cc(C)c1] | 1 mol % cat., 2 mol % B(C₆F₅)₃, 1 mmol formamide, 2 mL 1,4- dioxane, 4 bar NH₃, 150° C., 20 h | 16% |
| 16 | 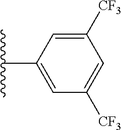 [SMILES: *c1cc(C(F)(F)F)cc(C(F)(F)F)c1] | 0.5 mol % cat., 1 mmol formamide, 2 mL 1,4- dioxane, 8 bar NH₃, 150° C., 20 h | 2% |
| 17 | R¹, R²: | 1 mol % cat., 1 mmol | 30% |
| | 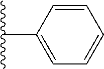 [SMILES: *c1ccccc1] | formamide, 2 mL 1,4- dioxane, 8 bar NH₃, 150° C., 20 h | |
| | R³: | | |
| | 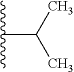 [SMILES: *C(C)C] | | |
| 18 | R¹, R²: | 0.2 mol % cat., 5 mmol | 6% |
| | 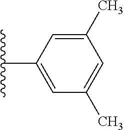 [SMILES: *c1cc(C)cc(C)c1] | formamide, 4.0 g NH₃, 150° C., 20 h | |
| | R³: | | |
| | 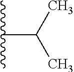 [SMILES: *C(C)C] | | |

### Examples 19 to 21

### Catalytic Activity of Ru-Phosphine Complexes as a Function of the Additional Ligands on Ruthenium (Nonphosphine Ligands)

The catalytic activity of various Ru-phosphine complexes in the synthesis of urea from formamide and ammonia was tested as a function of the nonphosphine ligands on the ruthenium. Table 3 indicates the complexes (catalysts) studied, the reaction conditions and the yields obtained. In the experiments the pressure was about 30 bar at the reaction temperature and the pressure in the cold state (room temperature) was about 8-10 bar. Example 19 corresponds to example 12.

Ruthenium-triphosphine complexes with the following structure were studied:

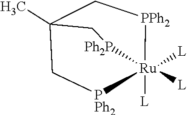

The three ligands L are shown in table 3 below, with one ligand L being designated L¹, a second ligand L L², and a third ligand L L³. In example 19 the three ligands L are formed together by the tridentate ligand trimethylenemethane (tmm). The pressures reported in the table relate to room temperature (about 23° C.). The autoclave was charged at room temperature and then brought to reaction temperature and reaction pressure.

TABLE 3

| Ex. | L = | Reaction conditions | Urea yield |
|---|---|---|---|
| 19 | L: | 1 mol % cat., 1 mmol | 64% |
| | 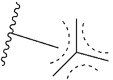 | formamide, 2 mL 1,4-dioxane 0.6 g NH₃, 150° C., 10 h | |
| 20 | L¹ = CO; | 1 mol % cat., 1 mmol | 18% |
| | L² = H; | formamide, 2 mL 1,4-dioxane, | |
| | L³ = Cl | 8 bar NH₃, 150° C., 20 h | |
| 21 | L¹ = CO; | 1 mol % cat., 1 mmol | 24% |
| | L², L³ = H | formamide, 2 mL 1,4-dioxane, | |
| | | 0.7 g NH₃, 150° C., 10 h | |

### Examples 22 to 28

### Catalytic Activity of Ru-Phosphine Complexes as a Function of Catalyst Concentration

The catalytic activity as a function of the catalyst concentration was tested for the following reaction conditions:

Catalyst: [Ru(triphos)(tmm)], 1 mmol formamide, 2 mL 1,4-dioxane, 0.6 g NH₃, 150° C., 10 h, with the catalyst concentration being varied. The reaction pressure was about 30 bar at the reaction temperature and the pressure in the cold state was about 8-10 bar.

Table 4 indicates the catalyst concentration (in mol % based on formamide) used under these reaction conditions, and the yields obtained.

TABLE 4

| Ex. | c(cat.) [mol %] | Urea yield [%] |
|---|---|---|
| 22 | 0.05 | <1 |
| 23 | 0.25 | 16 |
| 24 | 1 | 40 |
| 25 | 5 | 63 |

The catalytic activity as a function of the catalyst concentration was additionally tested for the following reaction conditions:

Catalyst: [Ru(triphos)(tmm)], 1 mmol formamide, 2 mL 1,4-dioxane, 4 bar NH₃ at room temperature (around 23° C.), 150° C., 20 h, with the catalyst concentration being varied.

Table 5 indicates the catalyst concentration (in mol % based on formamide) used under these reaction conditions, and the yields obtained.

TABLE 5

| Ex. | c(cat.) [mol %] | Urea yield [%] |
|---|---|---|
| 26 | 0.25 | 25 |
| 27 | 0.5 | 30 |
| 28 | 2 | 33 |

### Examples 29 to 35

### Catalytic Activity of Ru-Phosphine Complexes as a Function of the Solvent Concentration

The catalytic activity as a function of the solvent concentration was tested for the following reaction conditions:

Catalyst: 1 mol % [Ru(triphos)(tmm)], 1 mmol formamide, 0.6 g NH₃, 150° C., 10 h, with the solvent concentration being varied. The reaction pressure was about 30 bar at the reaction temperature and the pressure in the cold state was about 8-10 bar. The solvent was 1,4-dioxane.

Table 6 indicates the amount of 1,4-dioxane used under these reaction conditions, in ml (V(1,4-dioxane) [mL]), and the yields obtained.

TABLE 6

| Ex. | V(1,4-dioxane) [mL] | Urea yield [%] |
|---|---|---|
| 29 | 0.5 | 53 |
| 30 | 0.8 | 45 |
| 31 | 1.1 | 50 |
| 32 | 1.4 | 60 |
| 33 | 1.7 | 42 |
| 34 | 2.3 | 37 |
| 35 | 2.6 | 31 |

1.-15. (canceled)

16. A method of performing catalytic synthesis of urea, the method comprising using a ruthenium-phosphine complex as a catalyst.

17. The method of claim 16 comprising reacting formamide or formamide and ammonia in the presence of the catalyst to form urea and hydrogen.

18. The method of claim 16 wherein the ruthenium-phosphine complex comprises at least one monophosphine, one diphosphine, one triphosphine, or one compound having more than three phosphine groups, with the monophosphine having a formula PR¹R²R³ in which R¹, R², and R³ independently of one another are in each case substituted or unsubstituted alkyl, substituted or unsubstituted aryl, or substituted or unsubstituted heteroaryl.

19. The method of claim 18 wherein R¹ is alkyl and R² and R³ independently of one another are substituted or unsubstituted heteroaryl and/or substituted or unsubstituted aryl.

20. The method of claim 16 wherein the ruthenium-phosphine complex comprises one or more nonphosphine ligands that is selected from carbenes, amines, amides, phosphites, phosphoamidites, phosphorus-containing ethers or esters, sulfides, trimethylenemethane, cyclopentadienyl, allyl, methylallyl, ethylene, cyclooctadiene, acetylacetonate, acetate, hydride, halide, phenoxide, carbon monoxide, or a combination thereof.

21. The method of claim 16 wherein the ruthenium-phosphine complex comprises one or more nonphosphine ligands that is selected from trimethylenemethane, cyclopentadienyl, allyl, methylallyl, ethylene, cyclooctadiene, acetylacetonate, acetate, hydride, halide, phenoxide, carbon monoxide, or a combination thereof.

22. The method of claim 16 wherein the ruthenium-phosphine complex is a ruthenium-triphosphine complex, wherein the triphosphine has a general formula I: 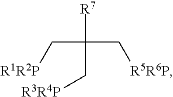
wherein R¹ to R⁶ independently of one another are substituted or unsubstituted aryl or substituted or unsubstituted heteroaryl, wherein R⁷ is hydrogen, alkyl, cycloalkyl, or aryl.

23. The method of claim 16 wherein the ruthenium-phosphine complex has a general formula II of (A)Ru(L)₃, in which A is a triphosphine of a general formula I: 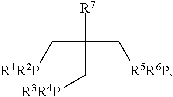
wherein R¹ to R⁶ independently of one another are substituted or unsubstituted aryl or substituted or unsubstituted heteroaryl, wherein R⁷ is hydrogen, alkyl, cycloalkyl, or aryl, wherein L in each case independently of one another are monodentate ligands, wherein two monodentate ligands L are replaceable by one bidentate ligand or wherein three monodentate ligands L are replaceable by one tridentate ligand.

24. The method of claim 17 wherein a concentration of the ruthenium-phosphine complex is in a range from 0.05 mol % to 10 mol % based on a molar amount of formamide.

25. The method of claim 16 wherein the catalytic synthesis is performed at a temperature in a 50 to 250° C. range.

26. The method of claim 16 wherein the catalytic synthesis is performed at a pressure in a range from ambient pressure to 150 bar.

27. The method of claim 16 wherein an amount of ammonia used in equivalents, based on formamide, is in a range from 1 to 300 eq.

28. The method of claim 16 wherein a reaction time of the catalytic synthesis is in a range from 1 minute to 24 hours.

29. The method of claim 16 wherein the catalytic synthesis is performed in one or more organic solvents or one or more ionic liquids.

30. The method of claim 16 wherein the catalytic synthesis performed in one or more organic solvents or one or more ionic liquids, with the solvent being cyclic and noncyclic ethers, substituted and unsubstituted aromatics, alkanes, or halogenated hydrocarbons, with the solvent being dioxane, 1,4-dioxane, toluene, or THF.

31. The method of claim 17 wherein the reaction of formamide or formamide and ammonia is a homogeneous catalytic reaction.

32. The method of claim 17 wherein the reaction of formamide or formamide and ammonia is a heterogeneous catalytic reaction.

33. The method of claim 17 wherein the reaction of formamide or formamide and ammonia is performed continuously.

34. The method of claim 17 wherein the reaction of formamide or formamide and ammonia is performed batchwise.

35. The method of claim 16 comprising using an acid as a cocatalyst in the catalytic synthesis.
